(19) United States
(12) Patent Application Publication
(10) Pub. No.: US 2020/0215109 A1
(43) Pub. Date: Jul. 9, 2020

(54) ACOUSTIC PROCESSING FOR CELL AND GENE THERAPY

(71) Applicant: FLODESIGN SONICS, INC., Wilbraham, MA (US)

(72) Inventors: Bart Lipkens, Bloomfiled, CT (US); Rui Tostoes, Northampton, MA (US); Walter M. Presz, JR., Wilbraham, MA (US); Benjamin Ross-Johnsrud, Northampton, MA (US); Kedar Chitale, Vernon, CT (US); Thomas J. Kennedy, III, Wilbraham, MA (US); Brian Dutra, Granby, CT (US); Brian McCarthy, East Longmeadow, MA (US); Jack Saloio, Ludlow, MA (US)

(21) Appl. No.: 16/244,100

(22) Filed: Jan. 9, 2019

(51) Int. Cl. A61K 35/17; B01L 3/00; C12M 1/00; C12N 13/00

(52) U.S. Cl. CPC A61K 35/17; B01L 3/502715; B01L 3/502761; B01L 2200/0652; C12N 13/00; B01L 2300/123; C12M 47/02

(57) ABSTRACT

A closed and modular fluidic system composed of one or more acoustic elements and cell processing reagents. The system employs a cellular manufacturing process for producing cell and gene therapy therapeutics.

### BRIEF DESCRIPTION OF THE SEVERAL VIEWS OF THE DRAWINGS

FIG. 1 is a high level block diagram of a cell therapy process.

FIG. 2 is diagram of an autologous cell therapy process.

FIG. 3A is a side elevation view of an acoustic module.

FIG. 3B is a cross-sectional side elevation view of an acoustic module.

FIG. 4 is a cross-sectional side elevation view of an acoustic module operated in a density gradient separation mode.

FIG. 5 is a diagram of a system for implementing a concentrate/wash operation.

FIG. 6A is a cross-sectional side elevation view of an acoustic module in a low cell density concentrate operation.

FIG. 6B is a cross-sectional side elevation view of an acoustic module in a low cell density wash operation.

FIG. 6C is a cross-sectional side elevation view of an acoustic module in a low cell density recover operation.

FIG. 7A is a cross-sectional side elevation view of an acoustic module in a high cell density concentrate operation.

FIG. 7B is a cross-sectional side elevation view of an acoustic module in a high cell density wash operation.

FIG. 7C is a cross-sectional side elevation view of an acoustic module in a high cell density recover operation.

FIG. 8 is a diagram of a system that includes beads for cell processing functions.

FIG. 9 is a diagram of an acoustic affinity separation system including a cross-sectional side elevation view of an acoustic affinity module.

FIG. 10 is two concentration graphs showing TCR+ cell concentrations.

FIG. 11A is a graph of TCR+ and TCR− cell concentrations in the absence of an acoustic filed.

FIG. 11B is a graph of TCR+ and TCR− cell concentrations in the presence of an acoustic filed.

FIG. 12 is two concentration graphs showing TCR− cell concentrations.

FIG. 13 is two graphs of TCR+ and TCR− cell distributions before and after acoustic processing.

FIG. 14 is a diagram of a system using a single acoustic module to perform multiple distinct operations.

FIG. 15 is a diagram showing cell and reagent colocation in the presence and absence of an acoustic field.

FIGS. 16A, 16B, 16C, 16D and 16E are graphs showing distributions under different acoustic settings.

FIGS. 17A and 17B are charts of results of different trials for acoustic transduction/transfection.

FIG. 18 is a graph showing distributions of transduction efficiency under different conditions.

FIG. 19 is a cross-sectional side elevation view of an angled wave device.

Like reference symbols in the various drawings indicate like elements.

### DETAILED DESCRIPTION

Cell therapy is a therapy that uses cellular material to treat a patient. Such therapy sometimes involves obtaining cells, which may be provided by the patient, modifying the cells for therapeutic purposes, and introducing the cells into the patient. The production process for obtaining a final product that is introduced to the patient involves a number of steps or processes for handling and/or manipulating the cellular material. This specification discusses a number of such processes that are implemented using acoustics to separate and/or fractionate and/or select materials and cells and/or retain materials or cells and/or manipulate cells or materials and/or culture cells. Cell therapy may involve processes such as bone marrow transplants.

Gene therapy involves introducing genetic material into the cell. The cellular and nucleus membranes are disrupted using techniques such as those based on chemical interaction, sonoporation, electroporation and/or other processes that allow for temporary gaps to be opened in the membranes. This disruption in the membrane allows for the introduction of genetic material such as nucleic acids into the cell.

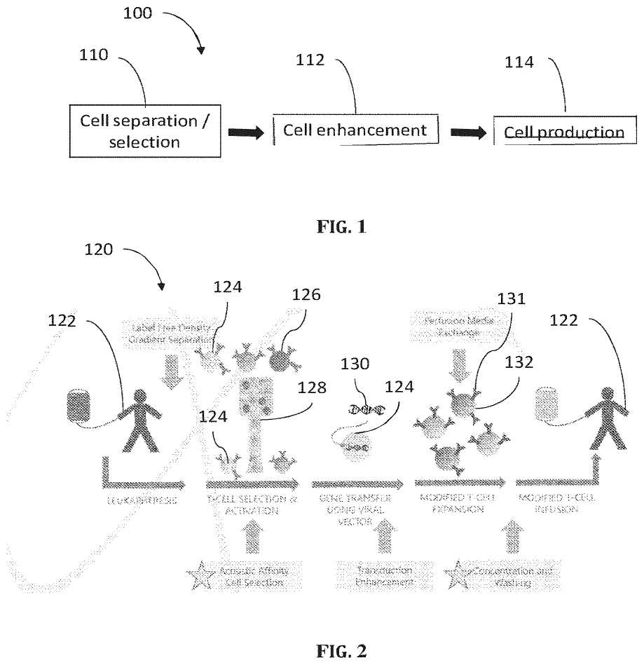

FIG. 1 shows a generalized cell therapy process 100. The cell therapy process 100 includes separating and/or selecting cells (step 110). In autologous cell therapy, the cells are obtained from the patient being treated. In allogeneic cell therapy, the cells are obtained from a source other than the patient being treated. Cells may be modified universally or specifically for cell therapy applications.

After specific cells are separated and/or selected, the cells are engineered and/or activated and/or expanded (step 112). For example, after a concentration and washing step, the genetic material of the cells can be modified by transduction or transfection. The cells can be cultured, and/or the cells can be differentially activated, genetically modified and expanded. A cell subtype can multiply and become dominant in the population. This result may happen as in cell type-specific activation such as T cell expansion, where T-cells are specifically activated by artificial antigen presenting cells (such as anti-CD3/anti-CD28 antibody-conjugated Dyna Beads) or by other means such as specialized material compounds or micro or nano beads. A process for generating chimeric antigen receptor T cells involves the steps of blood leukapheresis and T cell separation or the separation of T cells from a leukopak, T-cell activation by physical or material means, T-cell transduction utilizing a viral vector, T-cell expansion in a culture media and cryopreservation or direct administration to a patient. The T cells may divide multiple times in the in vitro culture as compared to the other peripheral blood mononuclear cells or may be enriched via metabolic selection, such as happens in the process of elimination of pluripotent stem cells via lactate accumulation, during cardiomyocyte differentiation.

Other techniques for enhancing and modifying cells for cell therapy include the use of Clustered Regularly Interspaced Short Palindromic Repeats (CRISPR), a family of DNA sequences that are utilized with CRISPR associated (Cas) genes that are located next to the CRISPR sequences. In particular, Cas9 (CRISPR associated protein 9) is utilized with the CRISPR DNA sequences. Other means of enhancing and modifying cells for cell therapy also include TALEN (transcription activator-like effector nuclease) and the Sleeping Beauty transposon system. Once the cell product has been enhanced, cell production can be used to produce large quantities of the enhanced cells (step 114). Acoustics can be used to perform some or all of these processes. For example, an acoustic cell culturing system can incorporate acoustic T-cell activation, acoustic transduction/transfection, and/or acoustic cell expansion. In some systems, the different steps are performed in different devices arranged in series. In some systems, the different steps are performed in series in a single device. It is understood that these processes occur in a fluid environment and thus may also be called acoustofluidic processes.

FIG. 2 schematically illustrates a T-cell therapy process 120 used to treat a patient 122 in which the patient's T-cells are engineered so they will attack cancer cells. FIG. 2 illustrates an autologous process in which the patient 122 is the source of the cells being enhanced and the recipient of the enhanced cells produced by the process. Similar processes can be used for allogeneic cell therapy. For example, T-cell therapy processes can be performed using a leukopak from other donors rather than blood directly from a patient as the source of the cells being enhanced.

Acoustic devices (e.g., label-free density gradient separation devices, angled wave separation devices, or angled flow separation devices) can be used to perform leukapheresis, the separation of white blood cells from a sample of the patient's blood and enhance the lymphocyte population?. The cells from the patient may also need to the unfrozen and separated from the cryogenic materials such as DMSO (dimethyl sulfoxide) before proceeding with the cell therapy process. After white blood cells are separated, the remainder of the patient's blood sample can be returned to the patient or discarded. The leukapheresis reduces the red blood cells (RBCs) and platelets present in the fluid being processed leaving primarily peripheral blood mononuclear cells (PBMC) such as, for example, lymphocytes (T cells, B cells, NK cells), granulocytes and monocytes. An example of acoustic density gradient separation is described in the discussion of FIG. 4. Examples of angled wave and angled flow separation systems are described in the discussion of FIG. 13. These systems use acoustic processes that differentiate the particles based on size, density, compressibility and/or acoustic contrast factor to separate components.

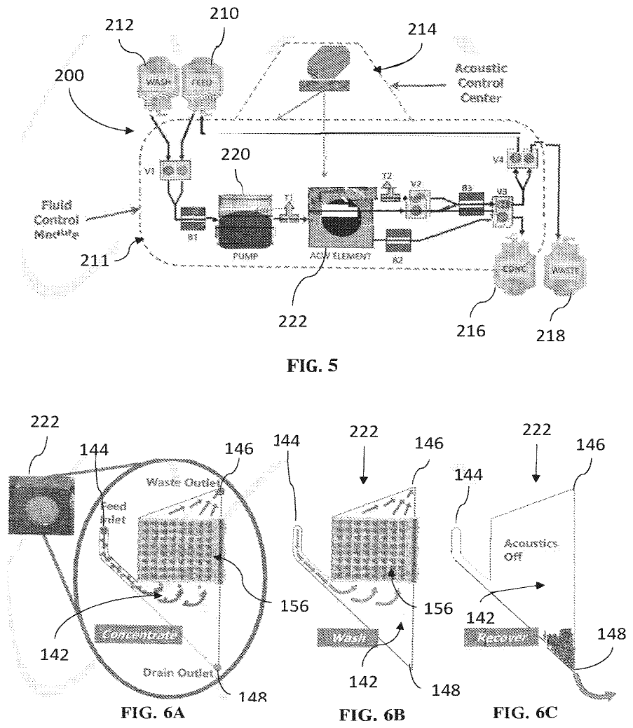

Between steps, a concentration/wash system can be used to process cells or target biomaterial to increase the concentration of cells in the fluid being processed, to remove undesired materials (e.g., non-target cells, cell fragments, platelets and debris), and to change the fluid carrying the cells. The T-cells may be washed and/or concentrated and/or washed, in different orders or to produce desired results for concentrate/wash operations. Some systems implement concentrate/wash operations using one or more acoustic devices that can retain the T cells and concentrate them into a reduced volume. Example concentration/wash systems are described in the discussion of FIGS. 5-8. Some methods and systems incorporate a concentrate/wash step after acoustic density gradient separation. For example, when a density gradient medium comprised of hydrophilic polysaccharides such as Ficoll-Paque™ is used for separation of a particular cell, e.g., RBCs, it may be necessary to wash out the remaining density gradient fluid in a subsequent process step.

The separated PBMCs are processed to select and activate specific type of T-cells. T-cells, also known as CD4+ or CD8+T lymphocytes, are a type of lymphocyte that plays a central role in cell-mediated immunity and can be distinguished from other immune cells by the presence of T-cell receptors on the cell surface. The T-cells include both target T-cells 124 and non-target cells 126

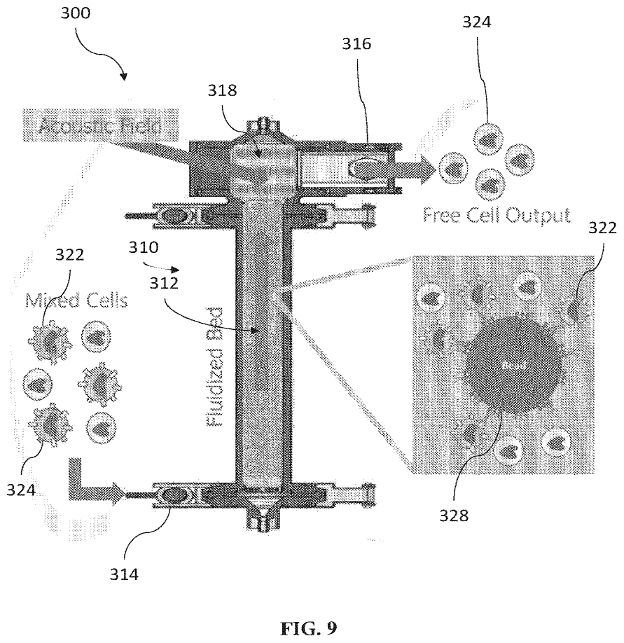

For example, an acoustic device 128 can be used to maintain microparticles, nanoparticles or micro-carriers (e.g., particles, beads, or bubbles) with an affinity for specific cells in a flow field. For example, the affinity selection process may implement selection based on markers such as, for example, CD3+, CD3+CD4+, and CD3+CD8+. The selection may also be utilized for the T-cell receptor selection, or TCR, that is a molecule found on the surface of T cells which is responsible for recognizing fragments of antigen as peptides bound to major histocompatibility complex (MHC) molecules. The acoustic affinity selection can be positive selection in which the micro-carriers have an affinity for the target T-cells or negative selection in which the micro-carriers have an affinity for non-target cells. The T-cell therapy process 120 uses negative selection with only the target T-cells passing through acoustic device 128. Some systems use positive selection to target CD3+ T-cells or subsets of CD3+ T-cells such as, for example, CD3CD4 and CD3CD8 T-cells or negative selection to remove monocytes and/or B-cells. Some methods and systems incorporate a concentrate/wash step after selection of specific cells to remove antibodies and other affinity selection reagents from the cell suspension and/or to concentrate the cell population for downstream applications. Some systems provide label-free selection of mononucleated cells (MNC) from the apheresis product. Example affinity selection systems are described in the discussion of FIG. 9.

After separation, the target T-cells are exposed to an activation reagent such as, for example, Dynabeads (Thermo) or TransAct (Miltenyi). These activation reagents usually contain antibodies specifically to T cell receptor and its co-stimulatory molecule CD28. After incubating T cells with these reagents ex vivo for hours or days, depending on the stimuli for activation. O, T cells divided multiple times and their number significantly expanded for later production process.

In one configuration of the T-cell therapy process 120, the activated T-cells are enhanced by using a viral vector to transfer genetic material 130 into the target T-cells 124 that enables the T-cells to express a chimeric antigen receptor (CAR) 131 on their outer surface that binds to a specific protein present on the patient's cancer cells. Although the T-cell therapy process 120 uses transduction (i.e., the process of introducing foreign DNA or RNA, depending upon the virus type, into a cell by a viral vector), some processes use transfection, electroporation or sonoporation, which do not require a viral vector to introduce foreign genetic material into a cell, or other processes to enhance the cells. In some systems, the gene transfer step is implemented with an acoustic process that traps and/or co-locates and/or concentrates the T-cells and, for example, a lentivirus or an adenovirus?.

In the T-cell therapy process 120, the population of modified T-cells 132 is expanded after enhancement. The expansion process can include a perfusion media exchange. Some systems implement the expansion process by culturing the cell population using an acoustic device that maintains the T cells in a culture in which the culture media may be exchanged throughout the culture period to add nutrients and cytokines (like glucose and interleukin-2) and to remove metabolic waste like lactate. After expansion, the modified T-cells 132 are concentrated and washed before being administered to the patient 122, for example, by infusion.

Some systems implementing the T-cell therapy process 120 are closed and modular acousto-fluidic systems with acoustic elements and cell processing reagents for a cellular manufacturing process on the scale of 30 to 150 billion cells and 750 mL to 5 L.

Some systems and methods implementing the T-cell therapy process 120 include mononuclear cell (MNC) isolation from apheresis products, isolation of T-cells (CD3+, CD3+CD4+ and CD3+CD8+ for instance) from apheresis products, removal of T-cell receptor positive cells (TCR+ cells) post cell engineering and expansion, as well as several wash and volume change steps.

Some systems and methods implementing the T-cell therapy process 120 include scale-dependent and/or scale-independent applications, or combinations thereof. Such implementations may control the cellular manufacturing process starting and final cell population and/or automate these process steps.

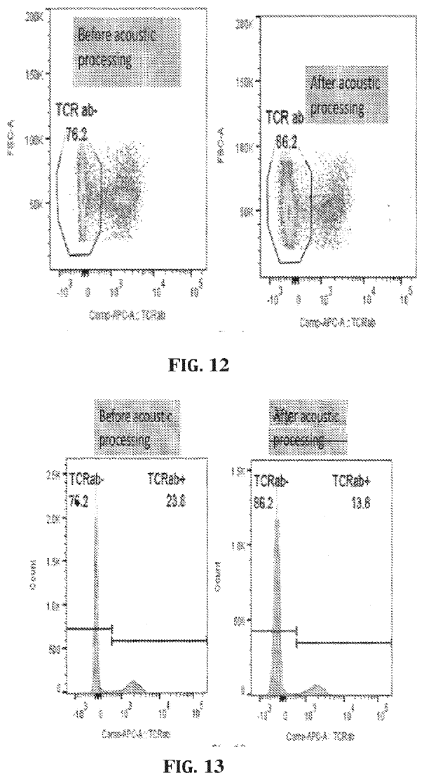

Some systems and methods implementing the T-cell therapy process 120 include one or more of the devices described with respect to FIGS. 5 to 14. These devices may be independent or integrated or combined in various combinations or sequences. Although generally described with respect to T-cell applications, other types of cellular material may be processed with these acoustic cellular processing systems and methods.

Acoustic Module

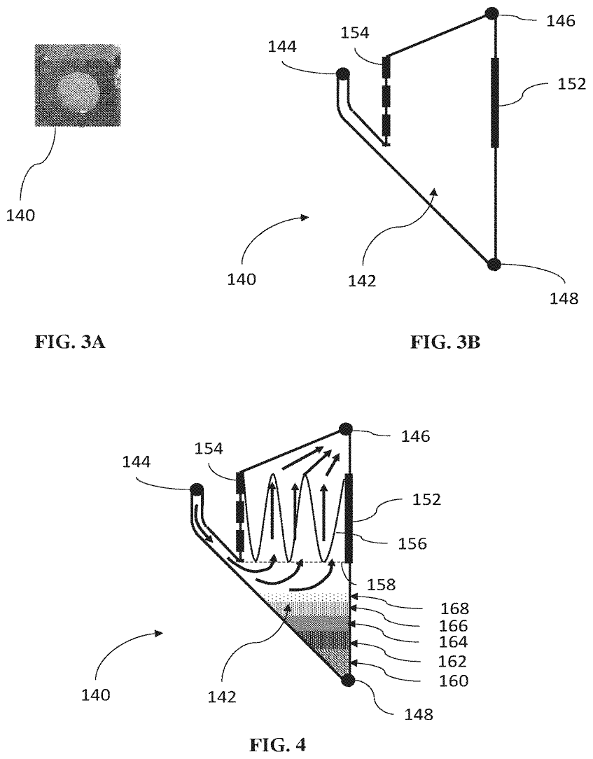

FIG. 3A and FIG. 3B, respectively, a photograph and a schematic of an acoustic module 140 that can be used to perform one or more steps such as, for example, acoustic density gradient separation, cell activation, concentrate/wash, gene transfer, and/or cell expansion steps of cell therapy processes such as those described with reference to FIG. 2.

The acoustic module 140 defines a flow chamber 142 with an inlet 144, an outlet 146, and a drain 148. A transducer 152 (e.g., an ultrasonic transducer) and a reflector 154 are positioned across the flow chamber 142 from each other. In some implementations, the reflector 154 is replaced by a second transducer 152. In operation, the transducer 152 creates an acoustic wave in fluid in the flow chamber 142. The acoustic wave interacts with the reflector 154 to create an acoustic standing wave. The transducer 152 can be operated to provide an acoustic standing wave creating an edge effect that limits entry of particular particles into the acoustic standing wave or to provide an acoustic standing wave creating a field of acoustic nodes and anti-nodes that capture particular particles within the acoustic standing wave. A prototype of the acoustic module 140 was constructed.

Acoustic Density Gradient Separation

FIG. 4 illustrates an acoustic module 140 being used for acoustic density gradient separation of white blood cells from other components of blood. Blood or diluted blood is pumped through the acoustic module 140 from the inlet 144 to the outlet 146 inducing the flow pattern indicated by the arrows in the flow chamber 142.

The transducer is operated to generate an acoustic standing wave 156 in the region between the transducer 152 and the reflector 154. For a particular type of operation, the system is typically tuned at a particular frequency, e.g., 1 or 2 MHz, to a particular value of the ratio of electrical power (in Watts) per unit flow rate (ml/min). Within a certain range, flow rate can be adjusted within the device, as long as the ratio of power per unit flow rate remains constant. Devices can be scaled up or down by changing the pathlength between transducer and reflector and by making the transducer and reflector wider. The scaled up or down device operates at the same linear velocity. The increase or decrease in flow rate is then given by the change in the cross-sectional area of the scaled device. Frequency of the standing wave is adjustable depending on the particle size of interest that is to be trapped in the standing wave. For cells, typical operating frequencies are between 500 kHz and 5 MHz. For smaller particles, e.g., viruses or exosomes, operating frequencies may be increased to 12 MHz, 24 MHz, or 36 MHz, or higher. For bigger affinity beads, operating frequencies may be lower, e.g., 100 kHz but can also be 1 or 2 MHz or higher. The acoustic standing wave traps cells of a certain size and acoustic contrast factor, e.g., RBCs 160 and WBCs, but may not trap platelets for a given set of operating conditions. Operating in a multimode pattern ensures that trapped cells cluster and settle out continuously when clusters reach a critical size depending on the properties of the fluid and cell. The collector is pre-filled with a density gradient medium 164 tuned to a density that is lower than that of RBCs 160 and granulocytes 162 but higher than that of PBMCs 166 such that RBCs 160 and granulocytes 162 settle through the density gradient medium 164 and fall to the bottom of the collector. The PBMCs 166 on the other hand will settle out of the acoustic field and settle on top of the density gradient medium 164 since their density is less than that of the density gradient medium. This layering effect will then allow for harvesting of enriched PBMCs 166. After the initial volume of blood has circulated through the device, the performance of the separation can be further increased by looping the outflow 146 back to the inlet back to the inlet 144 repeatedly so that over time the layering effect and density gradient separation is further enhanced. (Kedar, we have data on enhanced concentration which we should try to include here)

The acoustic standing wave 156 produces an edge effect creating a boundary 158 that limits or prevents the passage of particles. This effect retains RBCs 160, granulocytes 162, ficoll 164, PBMCs 166, and plasma 168 within the lower portion of the flow chamber 142. The flow velocity of fluid in this region of the flow chamber 142 is negligible and the retained components settle into discrete layers due to their relative densities.

The separation can be observed visually. After separation is complete, the different fractions are drawn off through the drain. After PBMCs cells are separated, the remainder of the patient's blood sample can be returned to the patient or discarded.

This approach applies much lower forces to the cells being separated than techniques such as, for example, counter-flow centrifugation.

Concentrate/Wash System

Physical means of concentration and washing, e.g., high-speed centrifuges, produce a large amount of stress and strain on immune cells such as, for example, T-cells, that may reduce the efficacy of the cells' immunological function. The acoustic module 140 described with reference to FIGS. 3A and 3B can use acoustic waves, including acoustic traveling waves and/or acoustic standing waves, to concentrate and/or wash immune cells. This approach provides a gentler process of concentrating and washing immune cells than by physical means. This approach has been shown to maintain high levels of cell health and/or viability.

Starting with an initial mixture that has a low cell density of, for example, less than 1×10⁶ cells/mL in an initial media, acoustophoresis can be used to reduce the volume of the initial mixture, for example by at least 10×, including 20× and up to 200× or more. The cell concentration may be increased by at least 10×, including 20× and up to 200× or more. The volume reduction factor is a function of the feed cell density. As feed cell density increases, obtainable volume reduction factors will decrease. As an example, at feed cell densities in the range of 20 to 40 million cells per ml, volume reduction can be 10×, including 20× and more. This initial reduction process is the first volume reduction step. Next, a second media (e.g., a biocompatible wash or buffer solution) can be introduced through inlet 144 and drain 148 to at least partially displace the first media and perform a washing step. Wash efficiencies can be 80%, 90%, 99% and more, depending on the amount of second media used. Next, the new mixture of the cells and second media can be subjected to an acoustophoretic volume reduction step. This series of operations is referred to as a “diafiltration” process. The range of cell concentrations and feed volumes that the acoustic concentrate wash device can handle is very broad; feed volumes can be as small as 200 ml and as large as 1000 ml, 3000 ml, 5000 ml and more; cell densities can as low as 150,000 cells per ml, can be 1-5 million cells per ml, 5-10 million cells per ml, 10-20 million cells per ml, and 20-50 million cells per ml. To obtain higher cell concentration in the collector, additional drain ports may be added so that the supernatant within the acoustic device can be removed.(need to add this possibility)

FIG. 5 shows an example concentration and washing system 200 including an acoustic device 222, sometimes referred to as an acoustic concentrate wash wave (ACW) element. The system 200 uses the acoustic module 140 (see FIGS. 3A and 3B) as the acoustic device 222. Some systems use other acoustic modules for their acoustic devices 222. Although described with reference to the concentration and washing of cells, the system 200 can be used to concentrate and/or wash other materials.

The acoustic device 222 is incorporated in a fluid control module 211 that also includes a number of switch valves V1, V2, V3, V4, a number of bubble sensors B1, B2, B3, and a number of Temperature sensors T1 and T2. A pump 220 is arranged upstream of the acoustic device 222 and configured or controlled to pump a fluid to flow through the acoustic device 222. In system 200, the pump is a peristaltic pump but some systems use other types of pumps such as, for example, a syringe pump.

The system 200 also includes an acoustic control center 214. In system 200, the acoustic control center is an integrated acoustic processing system configured to control the acoustic device 222 and the fluid control module 211 together. The acoustic control center 214 presents a graphical user interface (GUI), to a user, for controlling the acoustic device 222 and the fluid control module 211. Some acoustic control centers are implemented using other user interfaces. The acoustic control center 214 can provide automatic fluid flow by controlling the elements in the fluid control module 211, operates the various valves. The acoustic control center also maintains a certain operating point for the standing wave as needed, by automatically changing the frequency of excitation and the voltage signal to the transducer. It performs that function by continuously measuring the voltage signal across the transducer and the current going through the transducer. From these measurement, the control center calculates all transducer properties such as electrical impedance, resistance, reactance, real power, and apparent power. The same control center can be used to control any of the devices or processes disclosed.

In illustrated example, both feed fluid 210 containing the cells of interest and wash fluid flow 211 into the system 200 through the valve V1. In systems 200 in which the channels and flow chambers are provided by a sterile disposable cassette, it is not necessary to clean the system (e.g., with wash fluid) before use. In use, a wash fluid bag 212 and a feed fluid bag 214 are positioned above the fluid control module 211. This relative positioning allows gravity flow to prime the pump 220 when valve V1 is operated to provide a fluid connection between the wash fluid bag 212 or the feed fluid bag 214 and the pump 220.

After the pump 220 is primed, the fluid control module 211 is configured for concentration of cells contained in the feed fluid. The acoustic device 222 is controlled to generate acoustic waves in a flow chamber 142 of the acoustic device 222. Valve V1 is operated to provide a fluid connection between the feed bag 210 and the pump 220. Valve V3 is operated to isolate the drain outlet 148 and provide a fluid connection between the waste outlet 146 and valve V4. T2 is the temperature of the waste outlet and provides insight in any possible temperature rise across the acoustic field which may provide useful indication as to the successful operation of the system and making sure that cells do not experience any significant temperature rise Valve V2 allows switching between the waste outlet and the supernatant drain port. Valve V4 is operated to provide a fluid connection between valve V3 to a waste bag 218 or provide the option for recirculation of the waste outlet fluid back to the feed bag 210. At least two modes of operation exist. In a first mode, the feed fluid is recirculated for a fixed duration typically to establish cell clusters in the acoustic field which tend to increase the trapping efficiency of the system. At which point valve V4 is switched and the feed fluid is now emptied into the waste bag 218. This step continues until the bubble sensor B1 detects air at which point this process step is stopped. In a second mode recirculation may happen for the entire duration of this process step. In this mode, similar to diafiltration, cells are continuously trapped in the acoustic field, and the waste outlet containing fewer and fewer cells are sent back to the feed flow so that cells that escaped are passed through the acoustic field multiple times enhancing the probability of capture in the acoustic field. The pump 220 pumps the fluid to flow through the acoustic device 222 with a stable flow rate or with a varied flow rate. Flow rate is usually fixed during this process step. After a fixed time duration, the recirculation is stopped. At this point, the washing process is initiated by switching valve V1. The washing fluid flow can take on multiple fluid paths. Typically washing fluid flows in through the inlet 144 and collector drain 148 and in some embodiments additional wash ports are added. This is achieved through further valving (not shown, maybe we should show). The wash process takes place over a fixed time duration with a predetermined amount of wash fluid to achieve a desired washing efficiency such as 80% or 90% or 99% or more by displacing the feed fluid. The washing fluid is also discarded into the waste bag 218. When the washing process has ended, the pump stops the flow. At this point, flow has stopped. The acoustic field is then turned off and the trapped cells that have not settled out into the collector 142 yet are allowed to then settle into the collector 142. The settling process step is of a fixed duration as well, controlled through the control center. At the end of the settling process, valve V2 is switched and the supernatant is removed from the acoustic element and flown into the waste bag. (Don't we need a second pump?) (Or is the location of the pump correct?) The supernatant is the portion of the fluid volume 142 in the acoustic element above the collector volume that now contains all cells that have settled into the collector. Once the supernatant volume is removed, which is sensed through bubble sensor B3, valve V3 is switched and the removal of the concentrated and washed cells from the collector volume through drain 148 is initiated by the control center at some fixed flow rate. The concentrated and washed cells are flown into the concentration bag 216. Bubble sensor B2 is used as a sensor to determine when this process steps has completed.

FIG. 6A illustrates the acoustic device 222 during concentration of feed volumes at low cell concentration, may be 0.2 to 1 million cells per ml, or 1-2 million cells per ml. Concentrating can be achieved by means of capture or retention because of the much lower fluid volume of the ACW compared with the feed volume. As an example, typical ACW hold up volumes can be 15 ml, or 30 ml, or 80 ml, or more compared to feed volumes of 200 ml up to 5000 ml. With the fluid control module in the described concentration configuration, feed fluid containing the cells is pumped into the acoustic device 222 through the inlet 114, flows through the flow chamber 142 from bottom to top against the gravity, and out through the waste outlet 146. The acoustic waves can create a pressure field that generates primary and secondary acoustic radiation forces acting on the cells and cell clusters. The cells in the fluid can be held (or trapped) by the effect of the acoustic radiation forces. The fluid exiting of the acoustic device 222 flows through valve V2, valve V3, and valve V4 to the waste bag 218.

As the host fluid and material entrained in the host fluid flows upwards through the acoustic standing wave, the acoustic standing wave(s) traps (retains or holds) the material (e.g., secondary phase materials, including fluids and/or particles). The scattering of the acoustic field off the material results in a three-dimensional acoustic radiation force, which acts as a three-dimensional trapping field.

The three-dimensional acoustic radiation force generated in conjunction with an ultrasonic standing wave is referred to in this specification as a three-dimensional or multi-dimensional standing wave. The acoustic radiation force is proportional to the particle volume (e.g., the cube of the radius) of the material when the particle is small relative to the wavelength. The acoustic radiation force is proportional to frequency and the acoustic contrast factor. The acoustic radiation force scales with acoustic energy (e.g., the square of the acoustic pressure amplitude). For harmonic excitation, the sinusoidal spatial variation of the force drives the particles to the stable positions within the standing waves. When the acoustic radiation force exerted on the particles is stronger than the combined effect of fluid drag force and buoyancy and gravitational force, the particle can be trapped within the acoustic standing wave field.

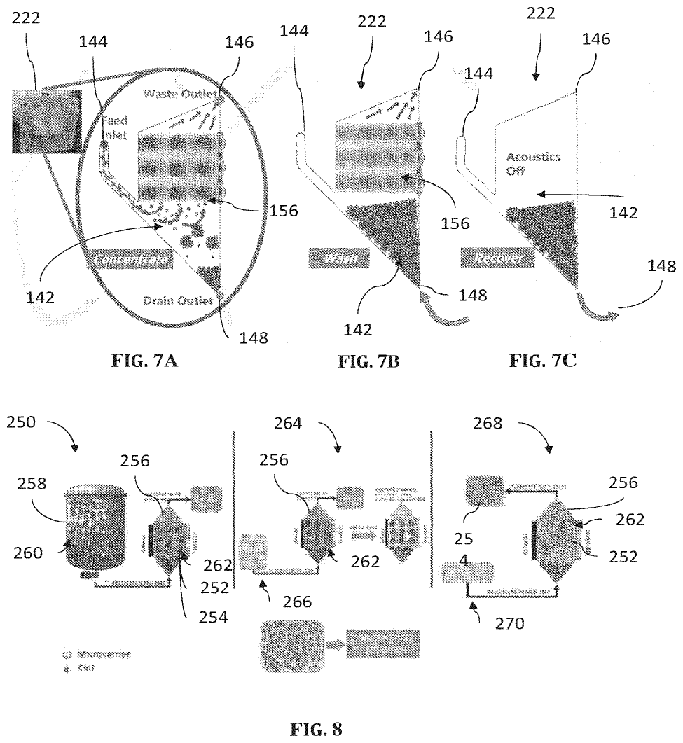

Desirably, the ultrasonic transducer(s) generate(s) a three-dimensional or multi-dimensional acoustic standing wave in the fluid that exerts a lateral force on the suspended particles to accompany the axial force so as to increase the particle trapping capabilities of the standing wave. A planar or one-dimensional acoustic standing wave may provide acoustic forces in the axial or wave propagation direction. The lateral force in planar or one-dimensional acoustic wave generation may be two orders of magnitude smaller than the axial force. The multi-dimensional acoustic standing wave may provide a lateral force that is significantly greater than that of the planar acoustic standing wave. For example, the lateral force may be of the same order of magnitude as the axial force in the multi-dimensional acoustic standing wave. A faceted reflector, or other shaped reflector, can be used to generate larger acoustic radiation force to further enhance the trapping strength of an acoustic field. A faceted reflector is shown schematically in FIG. 6A located opposite of the transducer. At higher cell densities flat or faceted reflector may be used. A flat reflector is shown in FIG. 7A opposite to the transducer.

After the cells are captured in the acoustic standing wave 156, the fluid control module 211 can be configured for washing the captured cells. The acoustic device 222 continues to be controlled to generate acoustic waves in a flow chamber 142 of the acoustic device 222. Valve V1 is switched to provide a fluid connection between the wash bag 212 and the pump 220. Valve V3 continues to isolate the drain outlet 148 and provide a fluid connection between the waste outlet 146 and valve V4. Valve V4 continues to provide a fluid connection between valve V3 to a waste bag 218.

FIG. 6B illustrates the acoustic device 222 during washing captured cells. With the fluid control module in the described washing configuration, wash fluid is pumped into the acoustic device 222 through the inlet 114, flows through the flow chamber 142 from bottom to top against the gravity, and out through the waste outlet 146. The fluid exiting of the acoustic device 222 flows through valve V2, valve V3, and valve V4 to the waste bag 218. The wash fluid can be the same type of buffer fluid that originally held the cells or can be a different type of buffer fluid. Although described as washing cells that have been concentrated, the washing process can be performed without a preceding concentration process. (as indicated above, concentration takes place because of the smaller hold up volume of the ACW. It could be done through a bigger unit) For example, the washing process can be used to change the buffer fluid containing a population of cells without reducing the volume of buffer fluid.

After concentration and/or washing, the fluid control module 211 is configured for recovery of the captured cells. The pump 220 is stopped and valve V3 is closed to isolate waste outlet 146 from downstream portions of the system 200. After the flow chamber 142 is sealed, the acoustic device 222 is deactivated. There is no flow of fluid in the flow chamber 142 and the cells previously captured in the acoustic standing wave 156 settle to the bottom of the flow chamber 142. The cells and a small volume of associated fluid is decanted from the drain outlet 148 of the flow chamber 142. Valve V3 is operated to provide a fluid connection between the drain outlet 148 and a concentrate bag 216.

In some implementations, the system 200 is configured to process fluids with low cell density, which can be used for buffer exchange for cell engineering. The low-density systems can be configured to provide throughput flow rates of some milliliter (mL) per minute (min) for fluids with 1-3 million (M) cells/mL feed concentration.

A prototype low-density system was constructed. The prototype system demonstrated the ability to concentrate cells while maintaining high cell viability. In one example, the prototype low-density system was used to concentrate and wash T-cells. Approximately 1 L of feed fluid with about 2 M cells/mL was processed in about 51 minutes. Table 1 shows the concentration data. The concentrated fluid had a very low final recovered volume 6.9 mL with a high final density of 250.7 M cells/mL. The viable cell recovery is about 84% with 160 times volume reduction.

TABLE 1
Primary T-Cell Concentration Data with Low Cell Density

| Process Parameter | Inputs | Outputs |
|---|---|---|
| Volume (mL) | 1105.8 | 6.9 |
| Viable Cell Density (M cells/mL) | 1.86 | 250.7 |
| Total Viable Cells (billion) | 2.06 | 1.73 |
| Cell Viability (%) | 99.1 | 97.9 |

In some implementations, the system 200 is configured to process fluids with high cell density, which can be used for buffer exchange for cell engineering.

FIGS. 7A-7C illustrate, respectively, the concentrate, wash, and recovery processes for a fluid with a high cell density. The same system 222 is used with different operating parameters. It can be operated with a flat or faceted reflector. These processes are substantially the same as those for low-density systems 222. However, the trapping of cells can result in clumping, and/or clustering of the trapped cells. Additionally, secondary inter-particle forces, such as Bjerkness forces, aid in cell clustering. As the particles continue to clump and/or cluster the particles can grow to a certain size at which gravitational forces on the particle cluster overcome the acoustic radiation force and fluid drag force. At such size, the particle cluster falls out of the acoustic standing wave.

During the concentration step, cells being captured in the acoustic standing wave 156 form clusters of cells through the action of all lateral forces and axial forces. Clusters of cells become large enough that gravity forces overcome the upward force of fluid flow through the flow chamber 142 and the trapping effects of the acoustic standing wave 156 and the clusters of cells settle to the bottom of the flow chamber 142 as shown in FIG. 7A. The frequency of cluster dropout is controlled by flowrate and cell concentration.

During washing, the feed inlet 144 is closed and wash fluid is introduced into the flow chamber 142 through the drain outlet 148 as shown in FIG. 7B. The flow rate is chosen to be low to avoid re-suspending the clusters of cells. Certain flow velocities are anticipated to be appropriate for cell clusters. For example, in the prototype system 222, a flow rate was used without significant re-suspension of cell clusters being observed. The acoustic device 222 continues to be controlled to generate acoustic waves in a flow chamber 142 of the acoustic device 222. The standing acoustic wave 156 limits or prevents cells or cell clusters that are re-suspended by the flow of wash fluid from being carrying out of the flow chamber 142 through the waste outlet 146.

During recovery, the waste outlet 146 is closed and the acoustic device 222 is deactivated. The cells and a small volume of associated fluid are decanted from the drain outlet 148 of the flow chamber 142 as shown in FIG. 7C.

In some implementations, the system 200 is configured to process fluids with high cell density. The high-density systems can be configured to provide throughput flow rates for fluids with 10-40 M cells/mL feed concentration.

A prototype high-density system was constructed. The prototype high-density system demonstrated the ability to concentrate cells while maintaining high cell viability. In one example, the prototype high-density system was used to concentrate and wash T-cells. Approximately 1 L of feed fluid with about 35 M cells/mL was processed in about 33 minutes without performing a washing step. Table 2 shows the concentration data. The concentrated fluid had a low final recovered volume 48.9 mL with a high final cell concentration of 587 M cells/mL. The viable cell recovery is about 86% with 19 times volume reduction.

TABLE 2
Primary T-Cell Concentration Data with High Cell Density

| Process Parameter | Inputs | Outputs |
|---|---|---|
| Volume (mL) | 949.9 | 48.9 |
| Viable Cell Density (M cells/mL) | 35.3 | 587 |
| Total Viable Cells (billion) | 33.5 | 28.7 |
| Cell Viability (%) | 98.8 | 98.0 |

The processing of the immune cells with the acoustic device 222 may include a single stage process/device and/or multi-stage process/devices, which may be used in the processing of the cell populations. The processes/stages may be single purpose or may integrate several steps in an overall immune cell processing system. The flexibility and potential for integration of steps can permit improved recovery of the cells that are being concentrated and washed.

In some implementations, the system 200 includes two or more low-density acoustic units coupled in series for multi-stage concentration and washing processes for fluids with low cell density. In some implementations, the system 200 includes two or more high-density acoustic units coupled in series for multi-stage concentration and washing processes for fluids with high cell density.

For example, a two-stage high-density acoustic unit system was modeled using two high-density acoustic devices in series without the other peripheral equipment required to provide a two-stage fluid control module prototype. In stage 1, a first fluid with a volume of 908.6 mL with 28.6 B cells (about 31.5 M cells/mL) was processed by a first high-density acoustic unit. The processing time was about 33 minutes. A concentrated fluid produced by the first high-density acoustic unit had a final volume of 48.9 mL having 23.6 B cells (about 482.6 M cells/mL). The viable cell recovery was about 83%. The waste fluid produced by the first high-density acoustic unit had a volume of 847 mL with 5.0?? B cells (about 8 M cells/mL). In stage 2, the waste fluid from stage 1 was processed by a second high-density acoustic unit. The processing time was about 33 minutes. A second concentrated fluid produced by the second high-density acoustic unit had a final volume of 50.8 mL with 3.3 B cells (about 65 M cells/mL). A second waste fluid flowed out of the second high-density acoustic unit had a volume of 790 mL having 3 B cells (about 3.8 M cells/mL). The viable cell recovery was about 48%. The first and second concentrated fluids were combined for a final concentrated fluid with a volume of 99.7 mL with 26.9 B cells (about 270 M cells/mL), and the viable cell recovery was about 94%. Thus, compared to a one-stage process, the 2-stage, in-series process achieved a higher viable cell recovery (about 94% in comparison to 83%) and more viable cells (26.9 B cells in comparison to 23.6 B cells).

In some implementations, the system 200 includes a combination of one or more low-density acoustic units for low cell density and low final volume and one or more high-density acoustic units for high cell density and high capacity. The low-density acoustic units can be coupled in series, the high-density acoustic units can be coupled in series, and the high-density acoustic units can be arranged downstream the low-density acoustic units.

The combination can be designed to be specific to different process ends. In some cases, the combination can be scaled down to decrease throughput, capacity, feed and/or final volumes, e.g., to 1/20 L units. In some cases, the combination can be expanded to increase throughput, capacity, feed and/or final volumes, e.g., to 5 L units or 20 L units.

During testing, it was also discovered that active cooling of the ultrasonic transducer led to greater throughput and efficiency and allowed a higher power delivery to the transducer. As such, a cooling unit was developed for actively cooling the transducer. The cooling unit includes an independent flow path that is separate from the flow path through the device containing the fluid that is to be exposed to the multi-dimensional acoustic standing wave. A coolant inlet is adapted to permit the ingress of a cooling fluid into the cooling unit. A coolant outlet serves as the outlet through which the coolant and waste heat exit the cooling unit. Here, the coolant inlet is located below the coolant outlet, though this path can be varied as desired. The coolant that flows through the cooling unit can be any appropriate fluid. For example, the coolant can be water, air, alcohol, ethanol, ammonia, or some combination thereof. The coolant can, in certain embodiments, be a liquid, gas, or gel. The coolant can be an electrically non-conductive fluid to prevent electric short-circuits. The cooling unit can be used to cool the ultrasonic transducer, which can be particularly advantageous when the device is to be run continuously with repeated processing and recirculation for an extended period of time (e.g., perfusion). The cooling unit can also be used to cool the host fluid running through the device, if desired.

FIG. 8 illustrates a four-step process (with an optional fifth step) for concentrating, washing, and separating microcarriers or other affinity beads, particles, or droplets from cells. The first step 250 in the process involves concentrating the microcarriers 252 with attached cells 254 in an acoustophoretic device 256. The microcarriers 252 and attached cells 254 can be introduced to the acoustophoretic device 256 by receiving the microcarriers 252 with attached cells 254 from a bioreactor 258. In the bioreactor 258, the microcarriers 252 and cells 254 are suspended in a first media 260 (e.g., growth serum or preservative material used to keep the cells viable in the bioreactor). The microcarriers 252 with attached cells 254 surrounded by the first media are concentrated by the acoustic standing wave(s) 262 generated in the acoustophoretic device. In a second step 264, the concentrated microcarriers 252 with attached cells 254 are then washed with a second media 266 to remove the first media 260 (e.g., bioreactor growth serum or preservative material). The third step 268 is to introduce a third media 270 containing an enzyme into the acoustophoretic device to detach the cells 254 from the microcarriers 252 through enzymatic action of the second media. In particular embodiments, trypsin is an enzyme used to detach the cells 254 from the microcarriers 252 enzymatically. The multi-dimensional acoustic standing wave 262 can then be used to separate the cells 254 from the microcarriers 252. Usually, this is done by trapping the microcarriers 252 in the multi-dimensional acoustic standing wave 262, while the detached cells 254 pass through with the third media. However, the cells can be trapped instead, if desired. Finally, the separated cells may optionally be concentrated and washed again, as desired.

After being concentrated and trapped/held in the multi-dimensional acoustic standing wave, the microcarriers can coalesce, clump, aggregate, agglomerate, and/or cluster to a critical size at which point the microcarriers fall out of the acoustic standing wave due to enhanced gravitational settling. The microcarriers can fall into a collector of the acoustophoretic device located below the acoustic standing wave, to be removed from the flow chamber.

During testing, steps one and two of concentration and washing, respectively, were performed using red and blue food dye to make colored fluid. The concentration mixture included SoloHill microcarriers in red fluid. The wash mixture included blue fluid and was passed through the device three times. The concentrate was observed under a microscope. The concentration step was shown to have a 99% efficiency. The first media (dyed red) was progressively washed out by a second media (dyed blue) over a series of wash passes. The light absorbance data is shown in Table 3.

TABLE 3
Light Absorbance

Light Absorbance

| Sample | Red (510 nm) | Blue (630 nm) |
|---|---|---|
| Feed | 0.138 | 0.041 |
| Wash Pass 1 | 0.080 | 0.066 |
| Wash Pass 2 | 0.063 | 0.080 |
| Wash Pass 3 | 0.054 | 0.084 |

The decrease in red light absorbance and increase in blue light absorbance evidences the feasibility of the washing steps. The testing of the acoustophoretic concentrating, washing, and separating process showed that the process is appropriate for cell therapy and microcarrier applications. The concentrate and wash steps were performed with a resulting efficiency of greater than 99%, and the separating step e.g., separating the cells from the microcarriers, was performed with greater than 98% efficiency. The cells had more than 98% viability.

Acoustic Affinity Separation System

FIG. 9 presents an example of an acoustic affinity separation system 300. As discussed with reference to FIG. 2, acoustic affinity separation systems can be used separate target cells (e.g., CD3+, CD3+CD4+, and CD3+CD8+ T-cells) from non-target cells and other material using positive selection or negative selection.

The affinity separation of biological materials, such as proteins or cells, is accomplished in some examples through the use of a ligand that interacts with a target biomolecule. This ligand can then be covalently or non-covalently attached to a surface such that the targetbiomolecule is captured. If the biomolecule is a transmembrane protein in a cell the whole cell will be captured by the affinity system.

A ligand is a substance that recognizes and forms a complex with the biomolecules. With protein-ligand binding, the ligand is usually a molecule which binds a specific site on a target protein which may be intracellular, extracellular or transmembrane; this binding may result in a change of conformation of the target protein, which in turn may produce a signal. The ligand can be a small molecule, ion, or protein which binds to the protein material. The relationship between ligand and binding partner is a function of charge, hydrophobicity, and molecular structure. Binding occurs by intermolecular forces such as ionic bonds, hydrogen bonds and van der Waals forces. The Association of docking is actually reversible through disassociation. Measurably irreversible covalent bonds between the ligand and target molecule is a typical in biological systems.

A ligand that can bind to a receptor, alter the function of the receptor, and trigger the receptor's physiological response is called an agonist for the receptor; a ligand that blocks the receptor's physiological response is an antagonist. Agonist binding to receptor can be characterized both in terms of how much physiological response can be triggered and in terms of the concentration of the agonist that is required to produce the physiological response. High affinity ligand binding implies that the relatively low concentration of the ligand is adequate to maximally occupy a ligand-binding site and trigger a physiological response. The lower the Ki level is, the more likely there will be a chemical reaction between the pending and the receptive antigen. Low-affinity binding (high Ki level) implies that a relatively high concentration of the ligand is required before the binding site is maximally occupied and the maximum physiological response to the ligand is achieved. Bivalent ligands consist of two connected molecules as ligands, and are used in scientific research to detect receptor dimers and to investigate the properties.

The T cell receptor, or TCR, is a molecule found on the surface of T cells or T lymphocytes, that is responsible for recognizing fragments of antigen as peptides bound to major histocompatibility complex (MHC) molecules. The binding between TCR and antigen peptides is of relatively low affinity and is degenerative.

The acoustic affinity separation system 300 includes an acoustic device 310 that can be operated to maintain (or retain) micro-carriers (e.g., particles, beads, droplets or bubbles) with an affinity for specific cells below an acoustic flow field by operating the acoustic field in the acoustic edge effect mode, also called acoustic interface effect mode, such that majority of the resin is held back by the acoustic field and are prevented from flowing into the acoustic field. The leading edge or interface of the acoustic field exerts a sufficiently strong downward force on the microcarriers to prevent them from entering the acoustic field. The microcarriers can be trapped in an acoustic field, such as a multi-dimensional acoustic standing wave or an edge effect discussed with respect to FIG. 4 can prevent the microcarriers leaving a flow chamber while free, non-bound cells may not be retained.

The acoustic device 310 has a flow chamber 312 with an inlet 314 and an outlet 316. The acoustic device is operable to generate an acoustic field 318 with an edge effect that limits the flow of resin out of the acoustic device 310. In this example, the microcarriers are microbeads 320 functionalized with ligands that preferentially bind to target cells 322. The interaction between the downward force of gravity and the upward force of fluid flowing through creates a fluidized bed of the microcarriers. The beads carry molecules for affine binding various targets with high specificity. Some of the affine molecules that may be used include antibodies, aptamers, oligonucleotides and receptors, among others. The targets for the affinity binding may include biomolecules, cells, exosomes, proteins, viruses, drugs, etc.

Although paramagnetic beads (e.g., iron or ferro-magnetic beads sold under the name Dynabeads or Miltenyi's . . . (find name)) have been used to achieve affinity extraction, the acoustic device 310 and similar devices enable affinity separation without requiring the beads of other microcarriers to be paramagnetic.

Non-magnetic beads with high acoustic contrast and affinity chemistry have been demonstrated. These acoustic beads can have functionalized material coatings or composition for affinity binding and are designed to be extracted from a complex mixture or fluid with an acoustic field. The acoustic beads can be directly used in applications developed in cell manufacturing, biochemistry, diagnostics, sensors, etc. that use magnetic beads. The acoustic beads can use the same surface and affinity chemistry as is used with magnetic beads. This ease of substitution of acoustic beads for magnetic beads has many advantages, including simplifying approval for applications, as well as simplifying the applications. One embodiment of affinity beads are liquid droplets of perfluorocarbon liquids such as perfluorohexane or perfluoro octylbromide (??). Such droplets are attractive affinity beads because of their high density (1.6 to 1.9 g/ml) and very low speeds of sound on the order of 400 to 600 m/s.

The acoustic beads can be made biocompatible. Such beads can be produced in different sizes, which permits continuous separation based on size in a size differentiating acoustic field, such as may be provided with an angled-field fractionation technology. The acoustic beads can be combined with an enclosed acoustics-based system, leading to a continuous end-to-end cycle for therapeutic cell manufacturing. This functionality provides an alternative to magnetic bead extraction, while preserving use of currently existing affinity chemistry, which can be directly transferred to the acoustic beads. The acoustic beads may be a consumable product in the separation operation.

In an example, a proof of concept trial was made using the published Memorial Sloan Kettering Cancer Center (MSKCC) protocol for extraction of CD3+ T cells from patient's blood. In the trial, paramagnetic beads were used, and the magnetic field is replaced with an acoustic field. The process of extracting CD3+ T cells from patient's blood is an integral part of manufacturing CAR (chimeric antigen receptor) T cells. Current processes are based on commercially available CD3 Dynabeads. In the trial, efforts were made to minimize the protocol differences, including performing the experiments in culture broth, rather than blood. The difference is considered reduced since several steps in CAR T cell manufacturing work from broth. The solvent density was increased to make T cells “acoustically invisible,” or not as susceptible to an acoustic field. The small size of the Dynabeads may provide an acoustic contrast that is similar to the cells, thus making separation tolerances smaller. The trial employed Jurkat CD3+ and CD3− T cell lines as models. The CD3− cells were employed as a control for non-specific trapping.

The cell suspensions were incubated with CD3 Dynabeads, which bound CD3+ cells. The mixture was passed through the acoustic system, which trapped the magnetic beads (with or without cells). The collected cells were successfully grown in culture. The cultured cells were examined with overlap of bright field images with fluorescence images. The beads were black with slight reddish autofluorescence. The live cells were fluorescent red. The bead diameter is 4.5 microns. CD3+ T-cell complexes with beads were observed, which demonstrates the efficiency of the technique. No CD3− T-cells were extracted in this example, which demonstrates the specificity.

In an example, a trial with acoustic beads was conducted. In this trial, agarose beads were used as the acoustic beads. These beads are available off-shelf from several manufacturers, and are not paramagnetic or have little to none iron or ferro magnetic content. Some agarose beads have surface modifications that simplify antibody attachment. They are also composed of biocompatible material, which can be important for therapeutic solutions. For example, ABT Beads, which are relatively inexpensive, heterogeneous (20-150 μm), off-shelf beads, which are available with streptavidin and biotin conjugates can be used. CellMosaic agarose beads, which tend to be relatively expensive, homogeneous (20-40 μm) can be configured with any modification by order.

The acoustic beads can be trapped in an acoustic field, such as a multi-dimensional acoustic standing wave. Proof-of-concept and validation of performance has been shown using acoustic affinity beads in an acoustic system. The disclosed methods and systems permit the use of off-shelf reagents, and currently available acoustic systems. The affinities can target any type of desired T cells or markers including TCR+, CD3+, CD4+, CD8+. The acoustic beads can have a high, neutral or low contrast factor, which can affect how the beads respond to an acoustic field, for example being urged toward an acoustic node or antinode, or passing through the field.

The beads may be composed of various materials and combinations, which permits development of optimal chemistry with acoustic performance and biocompatibility. The beads may be processed for isolation, sorting or any other function useful in a separation process. When used with a tuned acoustic system, the performance of specifically designed acoustic beads can match or exceed that of paramagnetic beads.

Existing chemistries may be used with the acoustic beads, and in conjunction with specifications of size and structure homogeneity to achieve desired results for acoustic and for isolation performance. The beads may be composed of composite constructs to advance acoustic efficiency. The acoustic system provides flexibility to manage small sizes, with heat management, and the use of fluidics to obtain results that are not possible with paramagnetic beads alone. The biocompatibility and/or biodegradability of the acoustic beads and simplified processing permits integration with existing hardware for CAR T cell manufacturing. The affinity acoustic beads can be used in a number of environments, including model environments such as, e.g., animal blood spiked with target cells and murine spleen extracts. The acoustic beads may thus be used in collaboration with existing systems, and may be designed and manufactured for target applications. The beads may be provided with a core that is acoustically active or neutral, and the bead themselves may be configured for high, neutral or low acoustic contrast. The size of the beads may be configured for separation and affinity in combination, for example a certain sized bead may include functionalized material to target a certain biomaterial, while another sized bead, may be functionalized to target another biomaterial, each of which can be separated simultaneously and continuously in a closed or flowing system. The beads can be designed to be of a homogeneous size distribution within a narrow or relatively broad range. Various affinity chemistries may be used, including streptavidin-biotin complex and immunoglobulin or aptamer. The beads may be designed for ease of manufacturability and/or for shelf-life. The beads may be used with approved chemistries, so that they may readily be integrated into known systems that use approved chemistries.

Affinity negative selection of TCR+ cells was demonstrated in an example trial with a volume of 1 L and 30 billion cells was specified. In a parallel trial, affinity negative selection of TCR+ cells with a volume of 5 L and 150 billion cells was demonstrated. Table 4 summarizes the results for the trials.

TABLE 4

| | | |
|---|---|---|
| Item | Baseline | Preferred |

| | | |
|---|---|---|
| Initial volume (flexible | 1 L | (5 L) |
| if FDS owns previous | | |
| stage of the process) | | |

| | |
|---|---|
| Final volume | 100-200 mL |
| | (500-1000 mL) |

| | | |
|---|---|---|
| Total viable cells | 30B | (150B) |

| | | |
|---|---|---|
| Viable TCR⁻ CAR⁺ cell | 70% | >70% |
| recovery | | |
| TCR⁺ cell removal | 99.9% | >99.9% |
| efficiency | | |

Affinity selection of CD3+ cells from an apheresis product was demonstrated in an example trial. Table 5 summarizes the results for the trial.

TABLE 5

| | | |
|---|---|---|
| Item | Baseline | Preferred |

| | | |
|---|---|---|
| Initial volume | 300 | mL |

| | | |
|---|---|---|
| Final volume | To be adjusted | |
| | for activation | |
| Total viable cells | 15B MNCs (correct | |
| | if T-cells) | |
| Viable CD3⁺ cell | 80% | >80% |
| recovery | | |

| | | | |
|---|---|---|---|
| Purity | 95% | CD3⁺ | >95% |

Affinity selection of CD3+CD4+ and CD3+CD8+ cells from an apheresis product was specified in an example trial. Table 6 summarizes the results for the trial.

TABLE 6

| | | |
|---|---|---|
| Item | Baseline | Preferred |

| | | |
|---|---|---|
| Initial volume | 300 | mL |

| | |
|---|---|
| Final volume | To be adjusted |
| | for activation |

| | | |
|---|---|---|
| Total viable cells | 15B | MNCs |

| | | |
|---|---|---|
| Viable CD3+ CD4+ | 80% | >80% |
| and CD3+ CD8+ | | |
| cell recovery | | |
| Purity | 95% CD3+ CD4+ | >95% |
| | and CD3+ CD8+ | |

Label-free selection of mononucleated cells (MNC) from apheresis product was demonstrated in an example trial. Table 7 summarizes the results for the trial.

TABLE 7

| Requirement | Baseline | Preferred |
|---|---|---|
| Initial volume | 300 mL | |
| Final volume | To be adjusted | |
| | for activation | |
| Total viable cells | 15B MNCs (correct | |
| | if T-cells) | |
| Viable MNC recovery | 80% | >80% |
| RBC, Platelets and | 99% | >99% |
| Granulocyte removal | | |
| efficiency | | |

The target T-cells separated by the processes described with respect to FIGS. 3A-9 are naive T-cells. After separation, the target T-cells are exposed to an activation reagent such as, for example, Interleukin-2 (IL-2), muromonab-CD3, TRANSACT T Cell Reagent commercially available from Miltenyi Biotec. Activation of the naive T-cells increases the division and proliferation rate of the T-cells and also triggers the differentiation of the T-cells (e.g., secretion of cytokines (helper cells), activation of killer functions (cytotoxic cells), acquisition of effector functions).

Acoustic Activation System

For example, activation of the T-cells can occur through the simultaneous engagement of the T-cell receptor and a co-stimulatory molecule on the T-cell by peptides and co-stimulatory molecules on an antigen presenting cell. Both are required for production of an effective immune response. The first signal is provided by binding of the T cell receptor to its cognate peptide presented on an antigen presenting cell (e.g., dendritic cells, B cells, and macrophages). The second signal comes from co-stimulation such as CD28, in which surface ligands on the antigen presenting cell are induced by stimuli (e.g., products of pathogens or breakdown products of cells, such as necrotic-bodies or heat shock proteins). The second signal allows the T cell to fully respond to an antigen presentation. Without the second signal, the T cell becomes anergic, and it becomes more difficult for the T-cell to be activated in future.

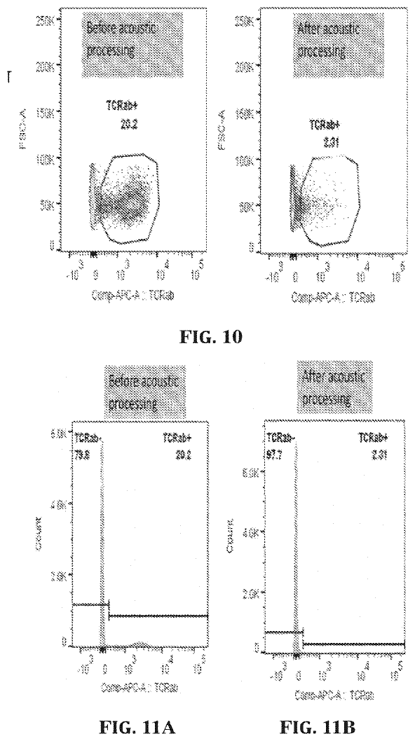

FIG. 10 illustrates a system 330 with a bioreactor 340 and an acoustic module 140. The system 330 can be used for transduction, transfection, activation, expansion/culture, concentration or washing of T-cells. The acoustic module 140 is fluidly connected with the bioreactor 340. A pump 342 pumps fluid from an outlet 344 of the bioreactor 340 to an inlet 144 of the acoustic module 140. Some systems locate pumps in other portions of the system. An inlet 344 of the bioreactor 340 receives fluid flowing out of the outlet 146 of the acoustic module 140. The bioreactor 340 has ports through which it receives, for example, culture medium from a reservoir 348, reagents (beads, antibodies), gases (e.g., oxygen, nitrogen, carbon dioxide) from a gas source 350 to maintain pH and dissolved oxygen. The bioreactor 340 includes a temperature control module 346 and a stirrer 348. In contrast to bioreactors that require heating to maintain desired temperatures of ˜36-37° C. for cell viability and growth, the bioreactor 340 includes temperature control module 346 that can heat or cool fluid in the bioreactor. The acoustic energy applied to the fluid by the transducer 152 tends to heat fluid in the system which reduces the required energy for heating and increases the need for temperature monitoring and control.

In operation, the pump 343 pumps culture medium from the bioreactor 340 into the acoustic module 140. The transducer 152 is operated to provide an acoustic wave that co-locates activation beads or reagents and cells in pressure nodes.

FIGS. 11A and 11B schematically illustrate the increased efficiency that this colocation is anticipated to provide assuming that high molecular weight reactions with cells are diffusion limited. Based on this assumption, the critical factors for activation include the diffusion rate and the binding rate. The diffusion rate is a factor of the fluid diffusion coefficient of the fluid; the molecular weight and diameter of the particles, cells, and reagents; the fluid temperature; and Reynolds number. The binding rate is intrinsic to reagents and cells.

FIG. 11A illustrates the spacing of cells and reagents in the absence of an acoustic field while FIG. 11B illustrates the spacing of cells and reagents in the presence of the acoustic field. High molecular weight reagents take longer to reach cell surfaces than low molecular weight reagents. Thus, the use of high molecular weight reagents requires longer incubation times and/or higher concentrations for the reagents to reach cell surfaces. However, the acoustic pressure nodes of an acoustic field will trap cells and attract higher molecular weight reagents. Secondary forces from cell clustering will enhance the trapping of high molecular weight reagents and also increase fluid viscosity at the node limiting reagent washout.

For a 1-inch flow chamber, flow rates of 1 liters per hour (L/h) produce a 2-4 centimeter per minute (cm/min) linear velocity between the transducer 152 and the reflector 154. These conditions provide low and controllable shear and stimulate cell aggregation that precedes and supports activation. The reagents are supplied at levels such as, for example, 3-4 activation beads/cell, 10 uL TRANSACT/million cells, or 0.5 μg anti-CD3/million cells. For most cell populations, the pH of the fluid is maintained between 6 and 8. Operating a bioreactor 340 with an acoustic module 140 for between 48 and 72 hours is anticipated to activate T-cells while amplifying input cell populations from 0.1-1 B total cells in to achieve 0.25-10 B total cells out. The process described with respect to FIG. 2 includes T-cell selection before activation. However, it is anticipated that T-cells should be dominant after activation even if the starting population is PBMCs rather than purified T-cells.

A prototype of the system 300 was tested with Human T-Activator CD3/CD28 DYNABEADS commercially available from ThermoFischer Scientific. The use of acoustics to control the activation beads enables the use of degradable, non-magnetic beads or other activation particles such as, for example, positive acoustic contrast, degradable beads made of poly(lactic-co-glycolic acid) (PLGA) containing IL-2 and/or other activation agents. These biologically compatible beads avoid the dangers associated with the possibility that metal-containing magnetic beads can be introduced into a patient with the therapeutic agents being manufactured by these processes. Some systems have bioreactors with volumes between 0.1 and 1 liter. After activation, the system 330 can also be used for washing the activated cells before and/or between transduction or transfection of activated cells and expansion of the enhanced cells.

Acoustic Cell Engineering

The system 330 can be used for performing transduction or transfection on the activated cells as described with reference to FIG. 2. After activation, the cells can be washed as described with reference to FIG. 6B. Transduction and transfection are performed using generally the same operational parameters as activation.

In transduction, 1 to 10 viral vectors/cell are added to the system and circulation is maintained for 24 to 48 hours. In a demonstration of transduction using the prototype system 330, RETRONECTIN was also added at a concentration of 4-20 μg/cm² after BSA non-specific blocking. As with the activation reagents, the acoustic field is anticipated to preferentially co-locate the viral vectors and the cells in pressure nodes. It is anticipated that replacing free viral vectors with positive contrast degradable beads containing a virus load will allow a tenfold reduction in the amount of viral vectors used as they will be concentrated in the nodes before release.

In transfection, 0.1 μg DNA/RNA is added per 0.1 million cells and circulation is maintained for 24 to 48 hours. As with the activation reagents, the acoustic field is anticipated to preferentially co-locate the DNA/RNA and the cells in pressure nodes. It is anticipated that replacing free DNA/RNA with positive contrast degradable beads containing a DNA/RNA load will allow a tenfold production in the amount of DNA/RNA used.

Higher frequency standing wave fields result in steeper pressure gradients which in turn are better suited for trapping smaller particles like viruses and DNA/RNA. Alternative materials (e.g., lithium niobate), fabrication methods (MEMs-based thick films), and specialized finishes (overtone polishing) are being used to create transducers operable to produce standing wave fields with frequencies between 0.01 and 100 MHz. These transducers will be easier to scale up than current transducers which are limited at higher frequencies and can be difficult to scale up to higher frequencies due to extreme thin thicknesses required (e.g., a 20 MHz transducer requires a 100 μm PZT element).

A prototype acoustic module 140 was used to demonstrate increases in transduction efficiency provided by an acoustic field. The effect of the acoustic module 140 on the transduction efficiency of baculoviruses used to modify Jurkat T-cells. Baculoviruses are rod-shaped, enveloped viruses of 30-60 nm in diameter and 250-300 nm in length.

FIGS. 11A-11E and Table 8 present the test results.

TABLE 8

| | Process | Process | Acoustic | Acoustic |
|---|---|---|---|---|
| Control | control 1 | control 2 | at 3 MHz | at 10 MHz |
| — | MOI: 50 | MOI: 50 | MOI: 10 | MOI: 10 |
| — | GFP+: | GFP+: | GFP+: | GFP+: |
| | 28.4% | 48.8% | 21.8% | 48.4% |

Acoustic Cell Expansion

The system 330 shown in FIG. 10 can also be used for expansion of the washed, enhanced cells. The expansion process can include a perfusion media exchange. Some systems implement the expansion process by culturing the cell population using an acoustic device that maintains or recycles the T cells in a culture in which the culture media is exchanged.

The enhanced cells can be kept in the same system 330 or transferred to another system 330 (e.g., a larger system). Prototype systems with 1 L and 5 L capacities have been produced. Systems have been designed with capacities between 0.5 L and 10 L. Operating bioreactor 340 with acoustic module 140 for between 8 and 12 days with a perfusion rate of between 0 and 2 volume of fresh medium/working volume of reactor/day (vvd) is anticipated to expand T-cells populations from the 0.25-10 B total cells produced by during activation to 10 B-100 B total cells out.

Angled Wave/Angled Flow Acoustic Cell Selection

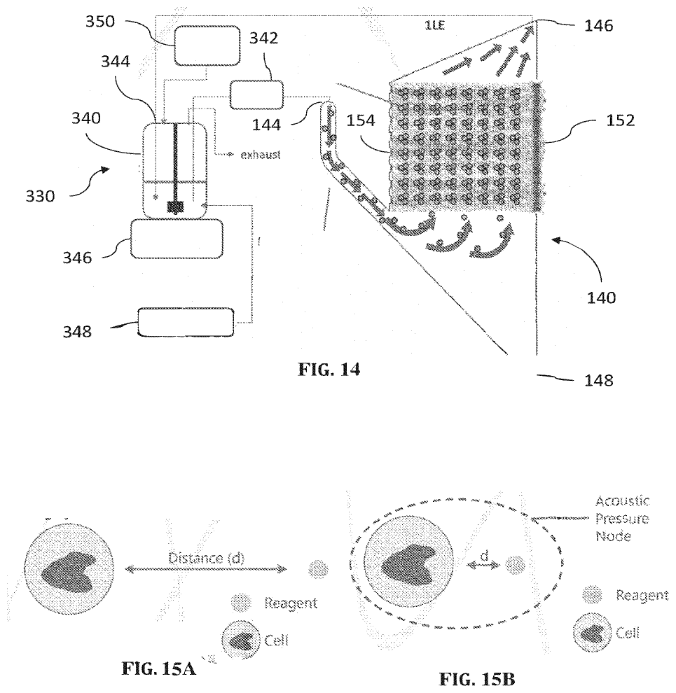

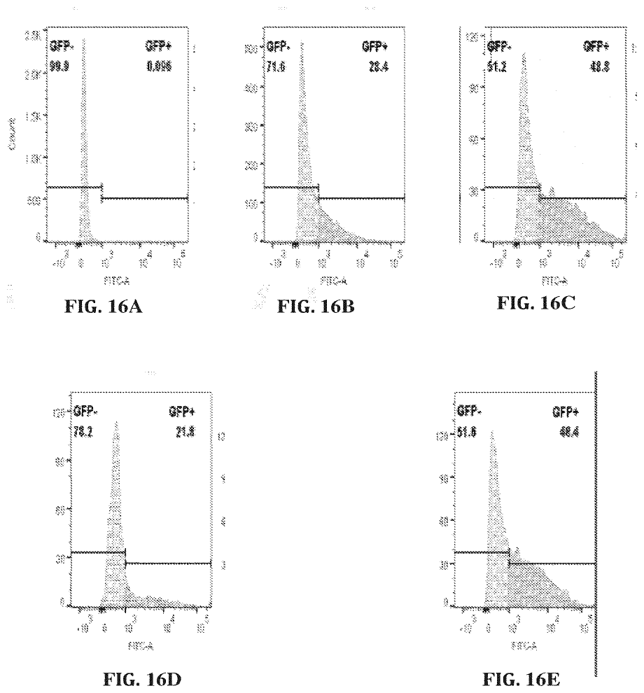

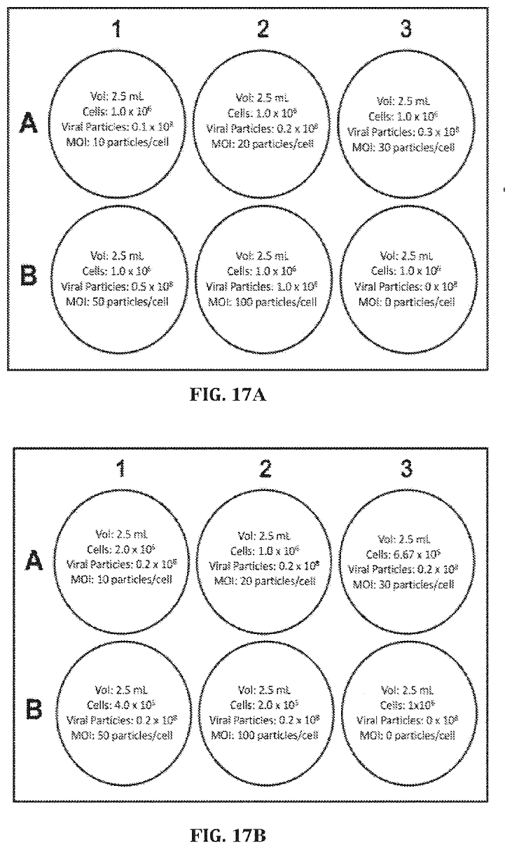

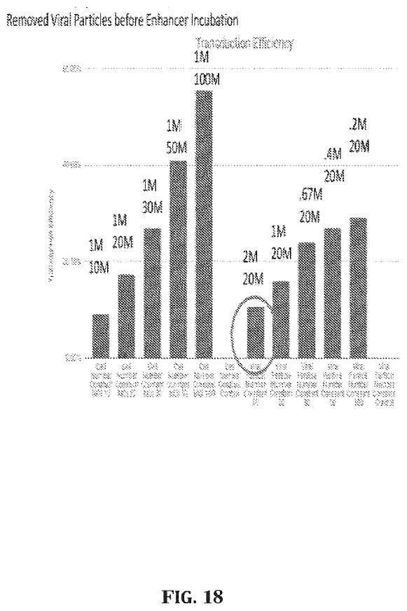

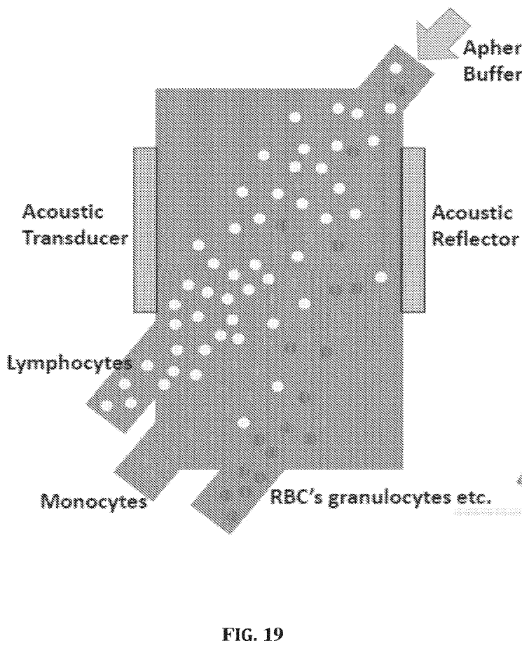

Other acoustic and non-acoustic modules can be used for some steps described with respect to FIG. 2. For example, angled wave or angled flow acoustic modules can be used instead of or in addition to the acoustic module 140 for RBC depletion and other fractionation processes. The fractionation of RBC, granulocyte, platelet and MNC using an angled wave device is discussed with reference to FIG. 14.

FIG. 13 illustrates an acoustic transducer that generates a bulk acoustic wave within a fluid flow with a mean direction flow that is angled relative to the acoustic wave. The angled acoustic wave can cause particles within the fluid to deflect at different angles that depend upon various characteristics of the particles. Thus, bulk acoustic standing waves angled relative to a direction of flow through a device can be used to deflect, collect, differentiate, or fractionate particles or cells from a fluid flowing through the device. FIG. 13 illustrates generation of angled acoustic standing waves due to the acoustic waves being reflected with the acoustic reflector. It should be understood that any type of acoustic wave may be used, including traveling waves, which may be implemented without an acoustic reflector, or maybe implemented with an acoustic absorber. The illustrated acoustic standing wave can be used to separate or fractionate particles in the fluid by, for example, size, density, speed of sound, and/or shape. The angled acoustic standing wave can be a three-dimensional acoustic standing wave. The acoustic standing wave may also be a planar wave where the piezoelectric material of the acoustic transducer is excited in a piston fashion, or the acoustic standing waves may be a combination of the planar acoustic standing waves and the multidimensional acoustic standing waves. The deflection of the particles by the standing wave can also be controlled or amplified by the strength of the acoustic field, the angle of the acoustic field, the properties of the fluid, the dimensionality or mode of the standing wave, the frequency of the standing wave, the acoustic chamber shape, and the mixture flow velocity.

When acoustic standing waves propagate in liquids, the fast oscillations may generate a non-oscillating force on particles suspended in the liquid or on an interface between liquids. This force is known as the acoustic radiation force. The force originates from the non-linearity of the propagating wave. As a result of the non-linearity, the wave is distorted as it propagates and the time-averages are nonzero. By serial expansion (according to perturbation theory), the first non-zero term will be the second-order term, which accounts for the acoustic radiation force. The acoustic radiation force on a particle, or a cell, in a fluid suspension is a function of the difference in radiation pressure on either side of the particle or cell. The physical description of the radiation force is a superposition of the incident wave and a scattered wave, in addition to the effect of the non-rigid particle oscillating with a different speed compared to the surrounding medium thereby radiating a wave.

As illustrated in FIG. 13, an apheresis product is fractionated into lymphocytes, monocytes and RBCs, granulocytes and other particles. This process can be used to isolate T cells in the apheresis product.

### Cell Therapy System—Example 1

### FIG. 14—Example—One Unit for Multiple Ops

14A—ACW in edge effect for density based separation—draw off RBCs 160, granulocytes 162, ficoll 164 leaving PBMCs 166, and plasma 168
14B—connect ACW to wash components
No selection—process all PBMCs
14C—Connect to bioreactor and activate, wash, transfect, expand

### FIG. 15—Example—Multiple Units in Series

15A—draw off and discard non-PBMCs, draw off and collect PBMCs
15B—transfer to concentrate & wash unit
15C—transfer to expanded bed for affinity selections of T-cells
15D—transfer to bioreactor unit for activation and enhancement
15E—transfer to larger bioreactor unit for expansion

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

What is claimed is:

1. A method for producing a therapeutic by implementing a series of processes, the method comprising:
obtaining cellular material suitable for invoking a therapeutic response;
wherein the processes include one or more of a process to concentrate the cellular material, a process to wash the cellular material, or a process for affinity selection of a portion of the cellular material; and
at least one of the processes employing an acoustic device to retain the cellular material or a structure to which the cellular material is bound.

2. The method of claim 1, further comprising:
fractionating the cellular material or the modified cellular material with an acoustic angled wave device.

3. The method of claim 2, wherein the cellular material is included in an apheresis product.

4. The method of claim 1, further comprising integrating one or more of the processes into a single device.

5. The method of claim 1, wherein the cellular material is housed in a bag.

6. The method of claim 1, wherein the series of processes form a closed system.

7. The method of claim 1, wherein the process for affinity selection includes negative selection for TCR+ cells.

8. The method of claim 1, wherein the series of processes form an end-to-end CAR T production process.

9. A system for producing a therapeutic by implementing a series of processes, the system comprising:
an acoustic device that includes an ultrasonic transducer configured to generate an acoustic wave to retain cellular material or a structure to which the cellular material is bound;
a chamber in the acoustic device for receiving the cellular material or a structure to which the cellular material is bound, the ultrasonic transducer being coupled to the chamber;
the acoustic device being configured to implement one or more of a concentration process, a washing process, or an affinity selection process.

10. The system of claim 9, further comprising an angled wave acoustic device for fractionating the cellular material.

11. The system of claim 10, wherein the cellular material is included in an apheresis product.

12. The system of claim 9, wherein the acoustic device is configured to integrate one or more of the concentration process, the washing process, or the affinity selection process.

13. The system of claim 9, wherein the cellular material is housed in a bag.

14. The system of claim 9, further comprising a closed system.

15. The system of claim 9, wherein the affinity selection process includes negative selection for TCR+ cells.

16. The system of claim 9, further comprising an end-to-end CAR T production process.

17. A cell therapy production system, comprising:
a number of interconnected devices that form a closed system, at least one of the devices being an acoustic device configured to retain cells or structures for supporting cells.

18. The system of claim 17, wherein the devices form and end-to-end cell therapy production system.
